(19) United States
(12) Patent Application Publication
(10) Pub. No.: US 2004/0225282 A1
(43) Pub. Date: Nov. 11, 2004

(54) Method and articles for assuring appropriate surgery

(76) Inventor: Anton P. Ness, Mechanicsburg, PA (US)

Correspondence Address: Anton P. Ness, 701 Colonial Court, Mechanicsburg, PA, 17050, US

(21) Appl. No.: 10/841,779

(22) Filed: May 7, 2004

Related U.S. Application Data
(60) Provisional application No. 60/469,097, filed on May 9, 2003.

(51) Int. Cl. A61B 17/00

(52) U.S. Cl. 606/1

(57) ABSTRACT

A concordance system for determining that an appropriate surgical or medical procedure is about to be performed on a patient, that has previously been authorized for that patient. The system includes visual inspection and scanning of preselected indicia on labels or cards respectively accompanying the patient, the designated surgeon, and the equipment or medical instruments and materials; and the scanning would be performed immediately prior to performance of a surgical procedure. The preselected indicia on the articles to be scanned or electronically read include: patient information, surgical or medical procedure name, surgeon identification, operating room identification, medical device or material or instrument or apparatus information. The scan or reading results when displayed would verify to the chief surgeon and others that all indicia are present and are consistent with one another. With this concordance system, the wrong operation would not have been performed on a patient, or the wrong medical device implanted thereinto, or the wrong materials used.

### RELATED APPLICATION DATA

This application claims priority from U.S. Provisional Patent Application Ser. No. 60/469,097 filed May 9, 2003.

### FIELD OF THE INVENTION

This relates to the practice of surgery or medical procedures, and more particularly to assuring that an appropriate scheduled and authorized surgical procedure is being performed on a patient.

### BACKGROUND OF THE INVENTION

It has been known to occur, that a surgical procedure is performed on a patient that is not appropriate for that patient. For example, an artificial knee is implanted in the left knee of a patient who should have had a right knee replacement, or where a right knee replacement is being appropriately performed on that patient but an artificial left knee is implanted, or nearly implanted, instead. Another example is that a left breast mastectomy is performed on a patient needing a right breast mastectomy. Yet another, more extreme example would be performing an angioplasty on a patient requiring removal of the gall bladder.

Many facilities (i.e., hospitals, medical centers, clinics) utilize their own particular verification procedures to assure that the operating room is carefully scheduled and is fully equipped with all necessary instruments, equipment, medical devices and medical materials, for the particular procedure to be performed on a given patient at a given date and time by a particular surgeon, and this verification comprises written and signed documents. Instrument trays or carts are prepared with all necessary instruments and materials for a particular patient's scheduled procedure following careful procedures, which tray is then covered to be kept sterile until transported to the designated operating room. Also, commonly, nurses or physicians even mark the patient manually with ink to designate the appropriate area of the patient involved in the surgical procedure and even may mark another part of the patient to indicate that surgery is not to be performed at that site; for example, the right foot is marked “yes” and the left foot marked “no” for right foot surgery.

Practically all medical facilities are believed equipped with computers, and information on patients, procedures, operating rooms, schedules, surgeons and inventory of all devices, materials, instruments and apparati are electronically stored and is readily accessible. Many new facilities or newly renovated facilities include premise wiring with ethernet-rated cable. And all medical devices, materials, instruments and equipment are required to be manufactured and properly labeled in accordance with requirements of the Food and Drug Administration in the United States. Yet, serious errors still occasionally occur despite all these precautions.

It is desired to provide a method to assure that only the surgical or other medical procedure is performed on a patient that is appropriate for that patient.

### SUMMARY OF THE INVENTION

The concordance coding system of the present invention provides for ascertaining that the appropriate patient is being prepared and presented for surgery, that the surgery being prepared to be performed is appropriate for that patient, that the correct medical device(s), material(s), instrument(s) or apparatus has been provided or prepared for that surgery, and that the designated surgeon for that patient is present to perform the authorized surgical procedure for that patient.

The concordance coding system preferably utilizes at least a visual and preferably a scanning method for scanning or machine-reading visible and/or scannable and/or machine-readable indicia on a selected suitable article such as a label or card, analogous to bar code scanning now commonly in use. The coding system could also utilize a magnetic strip or programmable microchip in a card insertable into a card reader, or one that may be used in conjunction with scanning; further, a diskette has relevant utility where a significant amount of data must be transferred in conjunction with this coding system. Most preferably, central to the coding system is a particular symbol, preferably one that uses a universally recognizable such as the venerable caduceous, located adjacent to alphanumeric and/or scannable or machine-readable indicia. Preferably, such symbol is asymmetrical in design for orientation of the scanned indicia, and preferably visually observable and recognizable, as well. The entire set of informational indicia may comprise at least four basic parts: the identification of the patient; the identification of the surgical procedure to be performed; the identification of the medical device or harvested organ, and/or materials, and/or primary surgical instruments or apparatus; and the identification of the particular performer of the surgical procedure. A fifth type of indicia could be included in the system, namely, the location where the procedure is to be performed and the date and time of the procedure. The five types of indicia can be preselectively located, respectively, to the left or right or top or bottom or central with respect to that symbol. With this concordance system, the wrong operation would not have been performed on a patient, or the wrong medical device implanted thereinto, or the wrong materials used.

The method of the present invention comprises at least the steps of: preparing at least a first article securable with the patient, containing scannable or machine-readable indicia at least identifying the patient and the authorized medical procedure; scanning or machine-reading the indicia at the immediate location of performance of the authorized medical procedure; and displaying results of the scanning or machine-reading to a medical practitioner authorized to perform the authorized medical procedure.

Optimally, the inventive method further includes the steps of: preparing at least a second article securable with the authorized medical practitioner, containing scannable or machine-readable indicia at least identifying the authorized practitioner, the patient and the authorized medical procedure; scanning or machine-reading said indicia at the immediate location of performance of the authorized medical procedure; and displaying results of the scanning or machine-reading and the said scanning or machine-reading of the first article to the medical practitioner authorized to perform the authorized medical procedure, to assure concordance of all relevant information on both the first and second articles.

Preferably, the concordance coding system utilizes a computer terminal to which the scanner/reader is coupled at the location of the procedure (i.e., the operating room). The computer is programmed not only to read and display the scanned/read indicia, but also to contain previously entered data regarding all the equipment and apparatus in that particular operating room, and all necessary items needed for each type of surgical procedure that could possibly be conducted in that operating room. The computer could be a stand-alone station with an array of CD-ROMs or diskettes for all surgical procedures, but could also access a server, if necessary. With use of this computer, the method would then include the steps of: previously entering information into the computer, the information including identifying all medical equipment located at the immediate location or brought to said location, and information associating said medical equipment to authorized medical procedures to performed at said location; and comparing entered information with scanned or machine-read information on the first and second articles.

An advantage of the present invention is that the surgeon observing the displayed results, can discover and correct discrepancies, such as replacing a certain instrument, or obtaining an additional, back-up item or material, especially if the computer is in communication with the hospital's inventory records, and proceed quickly with the surgery.

### BRIEF DESCRIPTION OF THE DRAWING

Embodiments of the concordance coding system of the present invention will now be described by way of reference to the accompanying drawing, in which:

FIG. 1 is a representation the entire set of concordance coding indicia;

FIG. 2 illustrates generally a scenario in which the concordance coding system would be used;

FIGS. 3 to 5 show examples of, respectively, a patient card, a surgeon card, and a scannable label (or card or diskette) for a medical device or instrument tray or the like; and

FIG. 6 diagrammatically illustrates the method by which the concordance coding system may be implemented.

### DETAILED DESCRIPTION OF THE PREFERRED EMBODIMENT

In a preferred concordance system of the present invention, the patient indicia can be located on a sticker or card or the like, such as to the left of a selected concordance code symbol. This indicia would be placed on an article secured to the patient such as a wrist band, or optionally a necklace or ankle band, or may be a card attached thereto, and would also include identification of the appropriate and authorized surgical procedure that has been approved to be performed on that patient, verification that the patient's informed consent has been obtained, and important patient information such as blood type (if relevant) or contraindications (if relevant). Appropriate contraindications could include known allergies to medications or limitations to alternative surgical procedures that may be used, if any, as restricted in the informed consent, such as restrictions on the anesthesia that may be used. The patient information could also optionally include the identification of the surgeon authorized or designated to perform the authorized surgical procedure, and also the particular operating room, date and time for the procedure. Preferably, the basic information on the patient's card is verified by preliminary scans at the patient's room or nurses station, or at the surgeon's office. A card form of article, similar to devices sometimes referred to as “smart cards,” can carry both visual and scannable information and also a magnetic strip and/or a microchip. Alternatively, or additional thereto, a removable biocompatible decal or sticker could be placed on the patient such as at a strategic location like the forehead, or at a location relevant to the particular procedure like left knee or right knee.

Further, the medical device, material, instrument or apparatus indicia could be located to the bottom of the selected symbol. This indicia would have been placed on a sticker on the medical device or at least its immediate packaging, or on the instrument or its container, or on the apparatus, and the particular surgical procedure for or with which it is designed or intended to be utilized. It would also include size or “left/right” designation, if relevant. Units of blood for transfusion, or containers for harvested organs for transplant, would of course contain at least blood type information. The indicia-containing article on an apparatus could constitute a sticker, which could be placed on newly received and on already existing apparatus by authorized personnel of the medical facility such as when the item is logged into inventory, or optionally by the manufacturer.

The practitioner indicia could be located on an additional article above the symbol. The indicia would be carried such as on an identification card, by the chief surgeon authorized to perform the scheduled surgical procedure, or an alternate practitioner, the surgical procedure to be performed, the patient on whom the surgical procedure is scheduled to be performed, and the location, date and time for that particular procedure. As with the patient card, the practitioner card could have visual and scannable indicia and/or a magnetic strip or microchip.

Location or operating room indicia would be located to the right of the symbol. Location information would constitute the particular operating room at which the particular procedure is to be performed, and the date and time scheduled.

In one preferred arrangement, the surgical procedure information can be located centrally with respect to the symbol, such as being overlaid or within the symbol or immediately therebelow. Preferably, the identity of the particular surgical procedure would be found on all of the aforementioned articles, assuring a high degree of integrity to the concordance coding system.

Presumably, every modern facility where surgical procedures are performed are equipped with a computer system and a local area network comprising multiple terminals and monitors. Also, such modern facility has a computerized inventory system continuously updated to identify all on-hand medical devices, materials, instruments and equipment, accounting for each such item when received and when materials and devices are used. Scheduling of operating rooms is also computerized to avoid conflicts and to select an operating room appropriate for particular types of surgical procedures.

It is most probable that the facility has data stored in its LAN regarding all possible surgical procedures. Included with such data would be every medical device, material, instrument and item of equipment necessary to each procedure, and also those common items that are useful therewith, and additionally, all other items that may be likely alternatives to such items. Data with respect to medical devices, materials, instruments and apparatus in inventory would include the size of the device and/or the “left” or “right” designation if relevant, or blood type of available units.

In one form, the patient card can originate as a card specific to as particular surgical procedure and containing all necessary information relevant to the procedure. When activated to be dedicated to a particular patient, all of the patient information could be entered onto or into the card by authorized facility personnel or by the authorized surgeon, thus transforming the procedure-specific card into a patient card. In such a form, the card could also serve to interrogate the computerized inventory facility to determine if all devices, materials, instruments or apparati are in stock or are otherwise readily available and usable as of the scheduled date of the surgical procedure, thus better assuring that all necessary (and possibly alternative) items would be present at the scheduled date, and can earmark them in the facility's inventory control system. Thus used, this activity would detect whether any item needs to be obtained by the facility, or even if a necessary item of unique equipment is scheduled to be used in another procedure at the same time and would thus enable rescheduling.

The operation of the concordance coding system will now be described. The quintessence of the system involves, immediately prior to performance of the surgical procedure, scanning and/or reading of the indicia of all concordance system-related articles brought to the operating room, and would be conducted at that operating room. If all elements comprising the total code are present and consistent with each other, both visual inspection and the scanning results would so indicate. In other words, “all must agree.” However, if not all articles are present at the operating room, or their indicia are inconsistent with each other, the scanner or reader, and even visual inspection, would indicate this situation; and correction can and should then be made or the surgical procedure should be halted.

The authorized surgeon, consistent with recognized prerogative, should be the appropriate person to ascertain and verify the results of the scanning of the code and thus authorize proceeding with the procedure. The surgeon would also have the authority to expressly override an element of the code, but such override should be documented. The surgeon, for example, could have an inappropriate or less preferred device or material removed and a proper one obtained, or could substitute an alternate medical device or instrument or apparatus, or select and prepare for an alternate surgical procedure should a complication arise. Following completion of the surgical procedure, the patient card and the performer card could be placed in the patient file of the facility, or the patient file of the surgeon. Also, the scannable label or diskette of the medical device(s) or material(s) could be placed in the patient's file as a permanent record, along with such information as the manufacturer's identification, any batch and lot numbers and the date of manufacture. This would serve to maintain such important information easily retrievable at a later date.

Preferably, a computer terminal is coupled to the scanner/reader at each operating room, that has already been programmed with all information concerning that operating room. Such information could include identification of all equipment and instruments permanently maintained in that particular room, and be updated whether or not the equipment and instruments are ready for used. Such permanent or portable equipment would include, for example, monitors, imaging equipment, anesthesia apparatus and fiberscopic or laparoscopic equipment. Further, the computer terminal has access to all information regarding particular surgical procedures, and the medical items associated with those procedures.

Commonly, the modern facility is large enough that instrument trays or carts are prepared at a site that is remote from the operating room, and sterility is assured during transport to the operating room at the appropriate time. Preferably with respect to the present invention, a readable article such as a diskette is prepared for each tray of instruments and materials for a particular surgical procedure, so that such article accompanies each such tray, or cart, when it is transported to the operating room. The article would identify each item on the tray or cart that is especially relevant to the particular procedure to be performed. One way of creating such an article would be for the personnel assembling the tray to enter into a computer the identity of each such item, most conveniently by using a touch-screen-input monitor that would then program the diskette.

Optionally, the nurse or physician's assistant in authority in the operating room for the particular surgical procedure could conduct a preliminary scan of the medical device(s), material(s), instrument(s), instrument tray(s) and apparati indicia for all items brought to the operating room for that procedure. For example, the nurse or assistant could thus assure that the appropriate medical device or instrument has already been obtained and is present on the tray or cart or is otherwise immediately available for use by the surgeon. The nurse could also preliminarily conduct a scan of the patient indicia when the patient has been brought to the operating room, to assure that the appropriate patient is present for the appropriate authorized procedure for which the operating room has been prepared. Further, the patient's card could be scanned prior to transport of that patient to the operating room to assure that all appropriate information has been entered onto the patient's card. These preliminary scans would thus serve to avoid delays in beginning the procedure.

Another aspect and benefit of using the present concordance system, would be evident when the patient is first transported to a pre-operation room. The patient card could there be scanned or read to assure that the preliminary procedures are performed and medication is provided, that is relevant and consistent with the authorized surgery or procedure to be performed immediately thereafter.

In preferred usage, the indicia for the medical devices, materials, instruments and equipment would be placed thereon by the facility upon receipt thereof and entry of the data into the inventory system of the facility. The indicia could be placed directly on the device or on its packaging, and on the packaging or container of the materials, or on the instrument or apparatus. Optionally, the indicia could be placed thereon by the manufacturer thereof, or by the facility where an implantable organ is harvested, or by the facility where blood is drawn. The patient indicia would be created by the admitting personnel in cooperation with the designated surgeon if relevant, and enhanced later by addition of the authorized surgical procedure, or blood type or informed consent or contraindications when determined or obtained later.

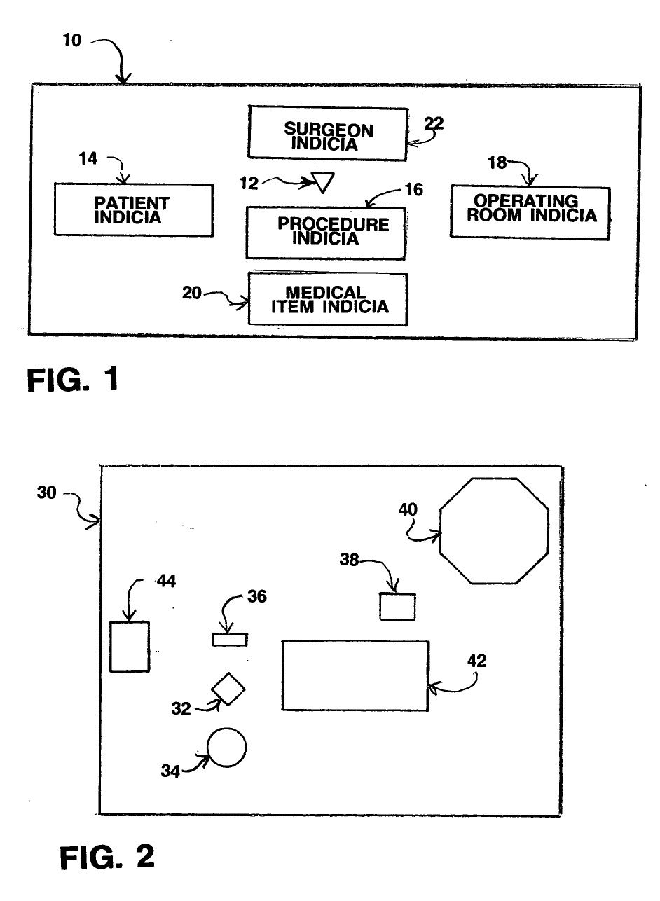

In FIG. 1, a representation of the entire set of concordance coding indicia 10 is illustrated in visual form. The central symbol 12 is shown as an inverted triangle but could in practice be or include a universally accepted symbol such as the caduceous. Patient indicia 14 is placed to the left of the symbol 12. Surgical procedure indicia 16 is located centrally with respect to the symbol adjacent thereto or overlaid thereon, and location indicia 18 to the right of the symbol. Medical device, material, instrument or apparatus (for convenience, termed “medical item”) indicia 20 is below the symbol, and surgeon or practitioner indicia 22 is located above the symbol.

FIG. 2 illustrates generally a simplified scenario in which the concordance coding system would be used. Shown are an exemplary operating room 30, medical device 32, medical material 34, instrument 36, surgical instrument tray or cart 38, apparatus 40, and patient table area 42. Scanning/reading/computer equipment 44 is shown in the operating room.

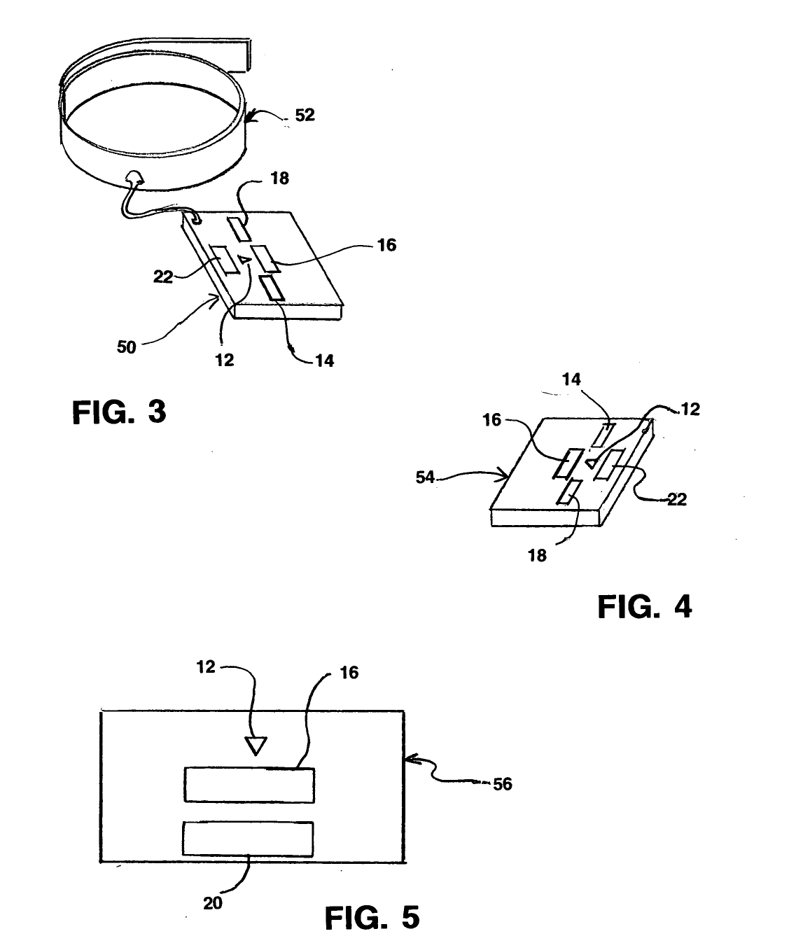

FIGS. 3 to 5 show examples of a patient card 50 attached to a wristband 52 adapted to be secured to the patient; a surgeon card 54; and scannable label (or card, or diskette) 56 for a medical device, instrument tray, or other medical item. Key indicia elements are shown designated thereon and preferably are visually recognizable, as well as scannable or readable.

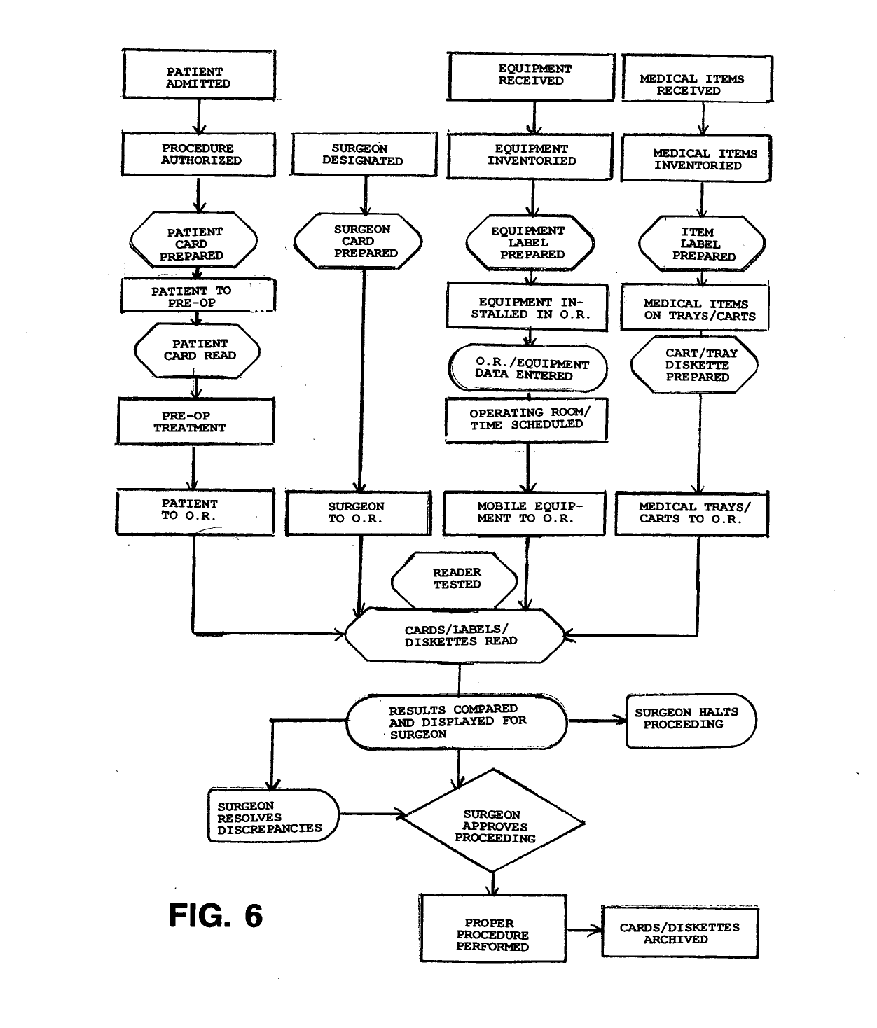

In FIG. 6 is illustrated the method by which the concordance coding system may be implemented. The facility can generate the article containing the medical item indicia onto the device, material, instrument or apparatus upon receipt thereof from the manufacturer or distributor; the article containing the indicia can be placed either on the device or on the immediate sterile packaging that encloses the device at all times prior to actual utilization in surgery and thereby be present in the operating room. For apparatus, such indicia could be affixed to already existing equipment and instruments in the manner of a permanent scannable label, decal or sticker. The source facility at which blood is drawn or the organ harvested, could perhaps create the labels for these items. The admitting facility would also generate an article (such as a diskette) containing instrument indicia when the tray of such instruments is being assembled in a sterile area in order to be transported to the operating room. The admitting facility would also create the article containing the patient indicia upon admission of the patient to the facility for performance of the scheduled surgery, and can complete or enhance this information on the article later when that information is determined or modified after admittance.

The circulating nurse can preliminarily scan or read the medical device, material, instrument, tray or apparatus indicia at the operating room upon setup immediately prior to the scheduled procedure. The designated or authorized surgeon can carry an identification card specific to the surgical procedure to be performed and the particular patient on whom the procedure is to be performed, and this would be scanned at the site of the procedure (i.e., operating room) when the surgeon arrives. It is preferable that the surgical procedure indicia be placed on all of the above articles: the patient's card, the medical device or material or instrument or tray or apparatus, and the designated practitioner's identification card.

The results of the complete visual inspection and optical scanning and electronic reading of all indicia would be ascertained and verified as positive by the designated surgeon and be formally documented. The designated surgeon would of course have the authority to override the scan results in the event of obvious malfunction of the scanning/reader equipment based on visual inspection of the articles, or where the scan results indicate an inconsistency between the various indicia or there is an understandable absence of a particular scannable article, but such override would of course be formally documented; for example, for certain instruments or devices or materials it may be awkward or impractical to include a scannable label thereon, but its precise identity is readily ascertainable by the surgeon and by the nurse.

Also, and preferably, a calibration test could be initially conducted by deliberately scanning or reading a test card or decal containing incorrect information, to deliberately achieve a negative reading, indicative of properly functioning scanner/reader equipment, whereafter the true or actual scan would be made.

Upon obtaining a negative result from the actual or true scan, the designated surgeon could at this time also arrange for obtaining an appropriate medical device, material or instrument or apparatus if such is not already present, or select an alternative device, material, instrument or apparatus. If a complication arises after a positive scan result has been obtained, that is, all indicia are in concordance, and verified and the procedure has been begun, the surgeon could even adopt an alternative procedure and be able to immediately ascertain from the already scanned patient indicia if that alternative procedure is expressly contraindicated. The designated surgeon could also select an alternative medical device, material, instrument or apparatus, if necessary. And, also, another appropriate surgeon could substitute were the originally authorized surgeon to have become unavailable, and this would be documented.

The benefits of the present concordance system are enormous, and the system itself is simple, not complicated, and relevant articles and software are well within presently known technology to be created inexpensively.

What I claim is:

1. An article securable with a patient upon whom a medical procedure is scheduled to be performed, said article including scannable or machine-readable indicia at least identifying the patient and the medical procedure.

2. The article of claim 1, further including a visually identifiable symbol selected to designate a pre-procedure concordance with a second like article containing complementary scannable or machine-readable indicia.

3. The article of claim 1, wherein said article includes a card adapted to be removably securable to the patient to assure arrival with the patient at the location for performance of the scheduled medical procedure.

4. An article securable with a medical practitioner authorized to perform a medical procedure upon a patient, said article including scannable or machine-readable indicia at least identifying the practitioner, the patient and the medical procedure to be performed.

5. The article of claim 4, further including a visually identifiable symbol selected to designate a pre-procedure concordance with a second, like article containing complementary scannable or machine-readable indicia.

6. The article of claim 4, wherein said article is a card accompanying the practitioner.

7. A medical procedure concordance method for assuring performance of an authorized medical procedure upon a patient, comprising the steps of:
preparing at least a first article securable with the patient, containing scannable or machine-readable indicia at least identifying the patient and the authorized medical procedure; and
scanning or machine-reading said indicia at the immediate location of performance of the authorized medical procedure; and
displaying results of said scanning or machine-reading to a medical practitioner authorized to perform the authorized medical procedure.

8. The method of claim 7, wherein said first article is securable to the patient in a manner permitting removal thereof from the patient after scanning or machine-reading at the immediate location of performance of the authorized medical procedure.

9. The method of claim 7, further including the steps of:
preparing at least a second article securable with an authorized medical practitioner, containing scannable or machine-readable indicia at least identifying the authorized practitioner, the patient and the authorized medical procedure; and
scanning or machine-reading said indicia at the immediate location of performance of the authorized medical procedure; and
displaying results of said scanning or machine-reading and the said scanning or machine-reading of said first article to the medical practitioner authorized to perform the authorized medical procedure, to assure concordance of all relevant information on both said first and second articles.

10. The method of claim 7, further including the steps of:
entering information into an apparatus located at the immediate location of performance of scheduled medical procedures, the information including identifying all medical equipment located at the immediate location or brought to said location, and information associating said medical equipment to authorized medical procedures to performed at said location;
comparing said entered information with scanned or machine-read information on at least a first article accompanying a patient arriving at said immediate location for performance of an authorized medical procedure thereupon, said first article including indicia at least identifying said patient and said authorized medical procedure; and
displaying the comparison results to an authorized medical practitioner to assure the practitioner that relevant medical equipment for performance of said authorized medical procedure is present at said immediate location.
